(19) United States
(12) Patent Application Publication
(10) Pub. No.: US 2023/0050346 A1
(43) Pub. Date: Feb. 16, 2023

(54) SYSTEM FOR TRACKING PATIENT REFERRALS

(71) Applicant: Jigneshkumar Patel, Trinity, FL (US)

(72) Inventor: Jigneshkumar Patel, Trinity, FL (US)

(21) Appl. No.: 17/818,803

(22) Filed: Aug. 10, 2022

Related U.S. Application Data
(60) Provisional application No. 63/309,189, filed on Feb. 11, 2022.
(60) Provisional application No. 63/231,505, filed on Aug. 10, 2021.

(51) Int. Cl. G06F 21/62; G16H 10/60; G06F 21/31

(52) U.S. Cl. CPC G06F 21/6245; G06F 21/31; G16H 10/60

(57) ABSTRACT

The instant invention relates to a system that in one form is a referral system for use in various healthcare sectors, which utilizes patient profiles, created using patient trackers, to monitor patient follow-up care. The instant invention allows physicians and providers to communicate about patient’s care in its entirety, beginning with the originating provider creating a patient tracker, sharing that patient tracker with referred physicians, and discharging the patient once the patient tracker’s plans have concluded. This uninterrupted flow of communication amongst all providers and physicians allows for a secured method of tracking each patient’s healthcare plan, provides for efficient healthcare referral follow-ups, and updates the originating HCPs records to ensure there are no gaps in terms of patient treatment.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### CROSS REFERENCE TO RELATED APPLICATIONS

"This application claims the benefit of U.S. Provisional Pat. Application Ser. No. 63/231505 filed on Aug. 10, 2021 and United States Provisional Patent Application Ser. No. 63/309189, filed on Feb. 11, 2022, both of which are incorporated by reference herein in their entirety.

### BACKGROUND OF THE INVENTION

### 1 Field of the Invention

The instant invention relates to a referral system structured to allow providers, patients and users to create patient profiles that enable providers to participate in patient tracking for healthcare related referrals to other physicians or providers within the instant invention network.

### 2 Description of Related Art

This disclosure relates to creating and managing complete patient profiles for healthcare treatment by various healthcare providers (HCPs), to bridge the gap between patients and providers, in order to ensure timely and effective patient care.

Therefore, what is needed in the art is a secured method of tracking a patient’s healthcare treatment plan and healthcare referrals, updating the originating HCP’s records and ensuring that there are no gaps in terms of patient treatment.

### BRIEF SUMMARY OF THE INVENTION

Embodiments of the instant invention can include mobile applications configured to interface with, but not limited to, smart phones, tablets, laptops, desktops, and/or mobile devices to allow users to interact with each other.

In one form the instant invention allows Health Care Providers (HCPs) the ability to seamlessly communicate among themselves using trackers for patient treatment and management. The system also facilities communication by providing a portal for secure messaging such as exchanging texts, images, videos and audio. The system provides a portal that facilitates notifications to patients and providers to improve patient follow-up and adherence to treatment. This eliminates the problem of keeping track of patients and the status of their follow-up treatment. The instant invention solves the problem of tracking a patient’s follow-up care, by creating HIPAA compliant communication between provider-to-owner, provider-to-provider, provider-to-ER and provider-to-lab/imaging and ensures that there is no communication gap between providers and physicians.

The instant invention in another form is a referral system structured to allow providers, patients and users to create patient profiles, for providers to participate in patient tracking for healthcare related referrals to other physicians/providers within the instant invention network. The system allows providers to invite other providers/physicians to participate in tracking patient referrals, update the status of patient procedures, and thereby notify the originating provider of the follow-up and discharge information for each patient. If a patient does not agree to be signed up for the HCP referral tracking, then they will not be allowed to participate thereby foregoing enhanced healthcare tracking. In at least one embodiment, a provider is required to be a member of the HCP network, but that may not be a requirement of all embodiments. The system is structured such that a provider will have a patient’s consent to sign them up on the instant invention, the provider will refer the patient to a member HCP, and the patient’s health history will be tracked using referrals until they are successfully discharged. The instant invention also uses different embodiments to track patient referrals and provider referrals. The instant invention is a mechanism to provide enhanced communications between various Healthcare Providers (HCPs) concerning a patient’s healthcare treatment, by allowing HCPs to create and manage a patient tracking system.

The instant invention in another form is a tracking system for use in patient healthcare between providers and physicians. It utilizes patient trackers to monitor important health information about patients. The instant invention also uses a HIPAA compliant text feature to communicate text, video, audio, and images between providers and physicians, to obtain updates on patient’s appointments, procedures, follow-ups and discharge dates. The inability to communicate a patient’s treatment plan and allow healthcare providers and physicians the ability to monitor their follow-up care, in real time, is an issue many healthcare providers currently face. The instant invention patient tracker system is structured to allow healthcare providers to bridge the gap in terms of patient treatment follow-up.

The instant invention has the advantage of bridging the gap between providers and physicians in order to ensure timely and effective patient care.

In a preferred embodiment the instant invention provides a secure method for patients and providers to provide information and share information. The most preferred embodiment is a platform that provides a common place to communicate ongoing care about an individual in a simplified way. The instant invention is divided into a Provider-to-Office (P20) module, a Provider-to-Provider (P2P) module, a Provider-to-Provider Office (P2P(O)) module and Provider-to-Provider Hospital (P2P(H)) module.

The system uses a security system to ensure that only authorized equipment can be connected to the system to provide the high level of security required by Health Insurance Portability and Accountability Act (HIPAA) regulations.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 shows a high-level flow chart of the provider-to-provider tracker.

FIG. 2 shows a flow chart that describes the method of the instant invention.

FIG. 3 shows the Web Application Architecture of the instant invention.

FIG. 4 shows the Provider & Surgery Center Login of the instant invention.

FIG. 5 shows the Provider-to-Office tracker of the instant invention.

FIG. 6 shows the screen that allows the provider to create a new Provider-to-Office Tracker of the instant invention.

FIG. 7 shows the screen that allows the provider to view Provider-to-Office Tracker of the instant invention.

FIG. 8 shows a screen showing the View Provider-to-Provider - Hospital Tracker of the instant invention.

FIG. 9 shows a screen showing the View Procedure(s) and Plan(s) screen of the instant invention.

FIG. 10 shows a screen showing the Add Procedure(s) and Plan(s) screen of the instant invention.

FIG. 11 shows a screen showing the instant invention ability to chat/texting with a provider.

FIG. 12 shows a screen showing the group tracker to add multiple providers.

FIG. 13 shows a screen showing how the instant invention creates a new provider-to-provider tracker.

FIG. 14 shows the mobile provider login in screen.

FIG. 15 shows the mobile screen for creating a Provider-to-Office tracker.

FIG. 16 shows the mobile screen for creating a Provider-to-Provider tracker.

FIG. 17 shows the mobile screen for creating the Chat or Texting with one or more providers.

FIG. 18 shows the mobile screen for View Provider-to-Providers information.

FIG. 19 shows the mobile screen to Add or Update Plan(s).

FIG. 20 shows the mobile screen to Add or Update Procedure(s).

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however, that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The use of the term physician and provider are used interchangeably to mean a professional user of the instant invention.

The use of the patient, user and client are used interchangeably to mean a patient user of the instant invention.

The use of the term application or app as used within the specification is meant to denote a computer application and such computer application can run on any smart device or computer.

The use of the term patient profile is meant to mean any patient specific information stored either electronically or in paper form.

The use of the term system, instant invention, and module is meant to mean any software system for users in a healthcare facility for making referrals and providing personalized follow-up care.

The use of the term tracker as used in the specification is meant to mean a software record that has patient and or provider information.

Currently the laws of the United States require that patient data and information stored or accessed by a computer system must be stored in a manner that prevents unauthorized access. The instant invention collects and provides access to patient data and therefore must provide a security system which is consistent with the rules mandated by the Health Insurance Portability and Accountability Act (HIPAA) regulations. Current password and username process can easily be overcome by sophisticated computer hackers and therefore the instant invention provides a novel third identification method for the providers and patients to ensure that individuals accessing the data stored on the system are authorized to access the data.

The instant invention allows providers to create login credentials comprising of a username, password and an NPI (National Provider Identifier) number. An NPI number is a 10-digit numerical identifier that identifies an individual provider or a healthcare entity. An NPI number is shared with other providers, employers, health plans, and payers for billing purposes, cell phones and office addresses and accept operating agreement and privacy policy. Once the login credentials have been created, the account will be approved by the administrator and the administrator will notify the user of their login credentials being verified. The instant invention stores the NPI number in a separate file to use as part of the login process. Those providers and facilities that authorize a number of users to view and enter data will be provided staff members and mid-levels team members to create sub-logins credentials under direct supervision of the provider. The system also captures the IP address and enters it in the sanction IP address file in the server/cloud application. Therefore, a provider must provide the NPI number any time a new device is used to access the system so the system can capture the IP address and enter it into the list of sanction IP addresses. The list of sanction IP addresses ensures that only sanctioned devices can communicate with the instant invention and have access to the HIPAA confidential information.

The instant invention software is available as an application for smart devices or desktop computer versions. The instant invention can provide both android and apple versions of the application as well as supporting other operating systems. The Portal patientcaretracker.com is the URL used to connect the user with the instant invention. A provider or the patient can access either a desktop or mobile version of the software using the same logins/credentials. As noted, the instant invention will also allow staff members and mid-levels to create sub-logins under direct supervision of the provider. There is an administrator account for each provider so that the various sanctioned devices and users can be monitored.

Currently in healthcare settings such as a healthcare facility and HCPs attend to the needs of their patients, prescribe medications, create treatment plans, and make referrals to various specialists and other physicians. While a patient is in the care of a HCP, every aspect of their treatment is closely monitored. The HCP monitor the patients to ensure that their medications are provided timely and on a schedule. The HCP also ensures that the various specialists visit the patient as required ensure they are in good health or recovering according to their treatment plans. Therefore, while in a facility the originating HCP is aware of the patient’s care and recovery on a daily basis.

When a patient is discharged from a healthcare facility, they are provided paperwork as part of their discharge papers, which may contain follow-up directives, such as specialists to contact or future follow-up doctor visits to monitor a patient’s progress. At this stage it is up to the patient to follow the prescribed treatment plan, schedule any appointments with recommended specialists, keep those appointments with the specialists, and provide any confirmation for the originating HCP, that the treatment plan was adhered to or that the treatment plan worked.

If a patient does not follow-up with the originating HCP or referrals to other HCPs as it relates to their healthcare, it is neither readily apparent to the originating HCP, nor readily communicated between providers and physicians. Although patients are not required to provide follow-up details to their medical providers, the lack of communication between providers and physicians creates a gap that affects the outcome of patient care. The instant invention seeks to bridge this communication gap between providers and physicians in order to ensure timely and effective patient care.

The instant invention allows HCPs to instantly communicate, in a secure and HIPAA compliant environment, about a patient’s care, about a patient’s progress concerning their injury or illness, and about a patient’s recovery. The instant invention allows the communication of messages sent and received to remain securely within the instant invention platform. One embodiment of the instant invention allows one provider to have one-on-one access to one other user, however other embodiments may allow for one provider to have access to more than one other user. For example, the originating provider creates a patient tracker in the instant invention and tags a first user for a first referral. No other users will be added to this patient tracker, and the secured communication concerning this patient, remains between this originating provider and the first user. If the originating provider makes a second referral to a different specialist, a new patient tracker must be created between the originating provider and this second user. Thus, one patient may have between one and any number of patient trackers that are active at any given time, depending on what their treatment plan requires. In a non-limiting example, if the patient’s treatment plan requires the patient seeing four different specialists, then four patient trackers would be created, one for each specialist. A specialist could be someone a patient is referred to, such as, but not limited to, physical therapy, ophthalmology, physiotherapy, and psychiatry.

P2O and P2P trackers are created using patient name, date of birth and phone number. Plan/procedures are medical specialty specific and can be entered in individual trackers. Each tracker has a chat box to send text messages including video and image. The instant invention can also create a group tracker to communicate among more than two providers. All information is password protected and encrypted for privacy reasons. A Provider can access the desktop version after entering password and One Time Pin (OTP) which is sent via text or email. This is done to provide increased security because of the sensitive HIPAA information stored in the instant invention. The Provider must use a unique 6-digit pin before login on a cell phone and use an application to view information. This uses an additional level of security to ensure that the mobile device is authorized because of the sensitive HIPAA information stored in the instant invention. The cloud application when receiving a request from a device checks the device IP address against a file which stores all the sanctioned IP addresses of devices which are allowed to have access to the sensitive data. If the IP address has a match, then the session is allowed.

The system security for access to or inputting of data is a critical business concern because of the requirements to comply with HIPAA regulations. The instant invention overcomes this problem of limiting information or access in multiple ways.

The instant invention also provides a patient profile application and portal to collect demographics, insurance and identification cards, six key health elements that includes past medical history, surgical history, family/social history, allergies and medication list, preferred pharmacy/lab/hospital and current providers (i.e., primary care physician and specialist such as a cardiologist of gastroenterologist. Patients also have access to their information in a secured manner when using their application on the desktop portal.

Patients have a 10-digit alphanumeric account number which is assigned by the system when they register and the system also captures their IP address for storage into the sanction IP address file associated with their 10-digit alphanumeric account number. The 10-digit alphanumeric account number can also used by the provider to access information for future visits to expedite care as well as collect and transfer accurate information.

The instant invention also provides software management service using account executives who can enter and maintain data. This is accomplished in HIPAA compliant manner using encrypted methods and trusted VPN and server services.

Additionally, other embodiments of the instant invention may allow for one provider to have access to more than one other user, decreasing the amount of patient trackers that are associated with one patient, and also allowing better communication concerning a patient’s overall care between a provider and a user.

Therefore, one can see that the intent of the instant invention is HIPAA compliant Healthcare application that offers a platform for providers to stay updated with patient’s movements in real time.

The instant invention can further be described as a HIPPA compliant tracking system for a patient’s follow-up healthcare services and monitoring system comprising a provider sign up module wherein the provider sign up module collects the provider login and security information and patient sign up module that collects the patient login and security information:
a. The instant invention also has a patient login module wherein the patient signs into the patient login module using the patient login and security information;
b. The instant invention also has a provider login module wherein the provider signs into the provider login module using the provider login and security information;
c. The tracking system collecting procedure information for at least one patient;
d. The tracking system collecting plan information for the at least one patient;
e. The tracking system displaying the procedure information for the at least one patient to at least one provider; and
f. The tracking system displaying the plan information for the at least one patient to at least one provider.

The provider tracking system wherein the provider login and security information comprises of a username, password, an NPI number and an IP address of the computing device the provider is using.

The provider tracking system wherein the provider login module compares the username, password, NPI number to authorized username, password, NPI number and the provider login module stores the IP address in a sanction IP address file if the login is a first time log in for the IP address if the username, password, NPI number are authorized.

The patient tracking system wherein the patient login and security information comprises of a username, password, 10-digit alphanumeric account number and an IP address of the computing device the patient is using.

The patient tracking system wherein the patient login module compares the username, password, 10-digit alphanumeric account number the authorized username, password, 10-digit alphanumeric account number and the patient login module stores the IP address in a sanction IP address file if the login is a first time log in for the IP address if the username, password, 10-digit alphanumeric account number are authorized.

The provider tracking system wherein the provider login module verifies the username, password, an NPI number and the IP address of the computing device and verifies that the IP address is in the sanctioned IP addresses and if the username, password, an NPI number is verified by the provider tracking system login verification system and the IP address is in the sanctioned IP addresses the provider tracking system login verification system allowing access to the provider tracking system.

The provider tracking system wherein the patient login module verifies the username, password, 10-digit alphanumeric and an IP address of the computing device and verifies that the IP address is in the sanctioned IP addresses and if the username, password, an NPI number is verified by the provider tracking system login verification system and the IP address is in the sanctioned IP addresses the provider tracking system login verification system allowing access to the provider tracking system.

The provider tracking system wherein the procedure information for the at least one patient comprises of a medical procedure, a provider, a facility and a date of service.

The provider tracking system wherein the plan information for the at least one patient comprises of a medical procedure, a provider, a facility and a date of service.

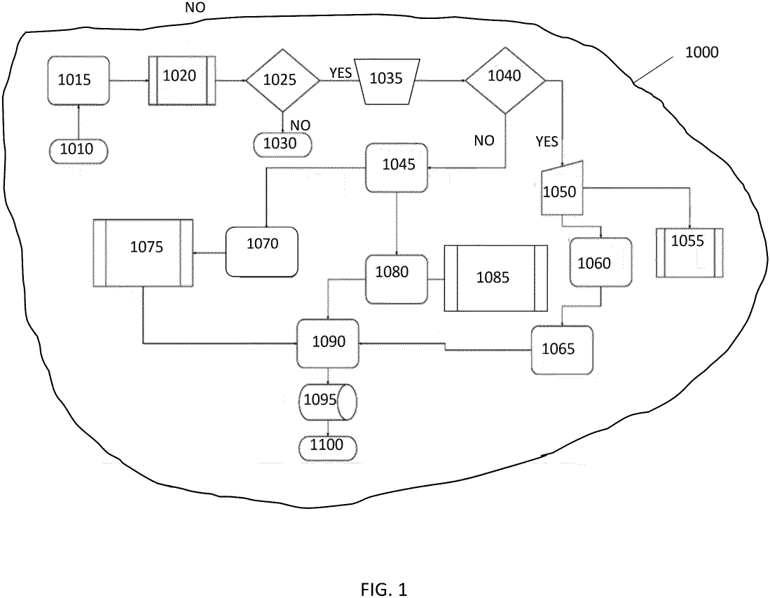

Referring now to FIGS. 1-20, and more particularly to FIG. 1, there is shown a high-level flow chart of the provider-to-provider tracker, one embodiment of the instant invention method 1000. At the start of the method step 1010 of the invention, the provider can login step 1015 to the patient tracker, where the system validates the provider/user step 1020. If that user is not an existing user step 1025, the provider’s attempt to login ends step 1030. If that user is an existing user step 1025, the provider selects the provider-to-provider tracker step 1035 and is presented with the option to create a new tracker step 1040. In creating a new tracker, the provider adds tracker details step 1050 which includes selected facility, name, phone number, date of birth, appointment date, follow up discharge date. The system saves the tracker in the list step 1055, a provider is assigned to a tracker step 1060 and the tracker is saved step 1065. If a tracker has already been created, the provider instead manages the existing tracker step 1045 which manages notes, patient details, provider information and messages. The instant invention then is able to send messages to providers for selected patients step 1070, and those messages are sent and received following compliance rules step 1075. The provider also manages plans and procedures for patients step 1080 where the instant invention can add, edit, and update plans and procedures. Each new event created is time stamped for each plan and procedure step 1085. The data is then encrypted step 1090 and stored step 1095 and the method of the instant invention ends 1100.

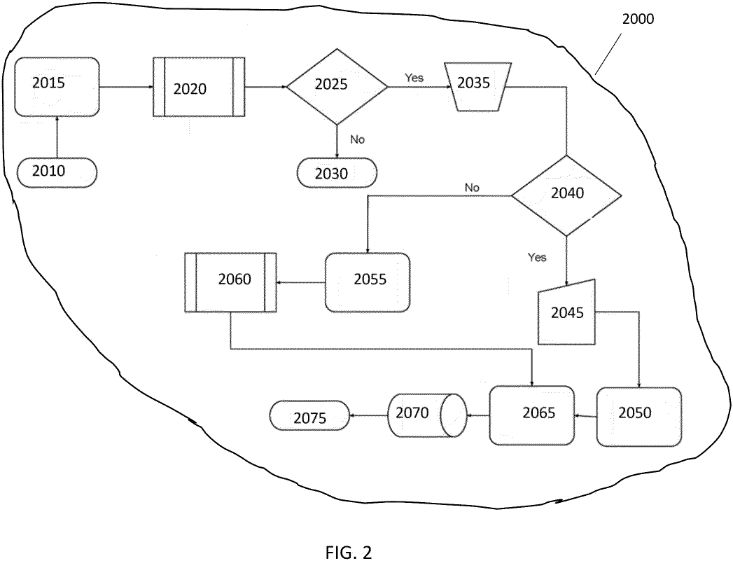

Referring to FIG. 2, there is shown method step 2000 of the instant invention. At the start of the method step 2010 of the invention, the provider can login step 2015 to the patient tracker where the system validates the provider/user step 2020. If that user is not an existing user step 2025, the provider’s attempt to login ends step 2030, and if that user is an existing user step 2025, the provider selects the Provider-to-Office tracker step 2035 and continues to create a new tracker step 2040. The provider adds tracker details step 2045 such as select facility, name, phone number, Date of birth, appointment date, follow up date and discharge date. The tracker is saved step 2050. If a tracker has already been created, the provider manages the existing tracker step 2055 where the instant invention can manage notes, patent details, follow up and discharge details. The system saves the updated information step 2060. The data is then encrypted step 2065 and stored step 2070 and the method of the instant invention ends step 2075.

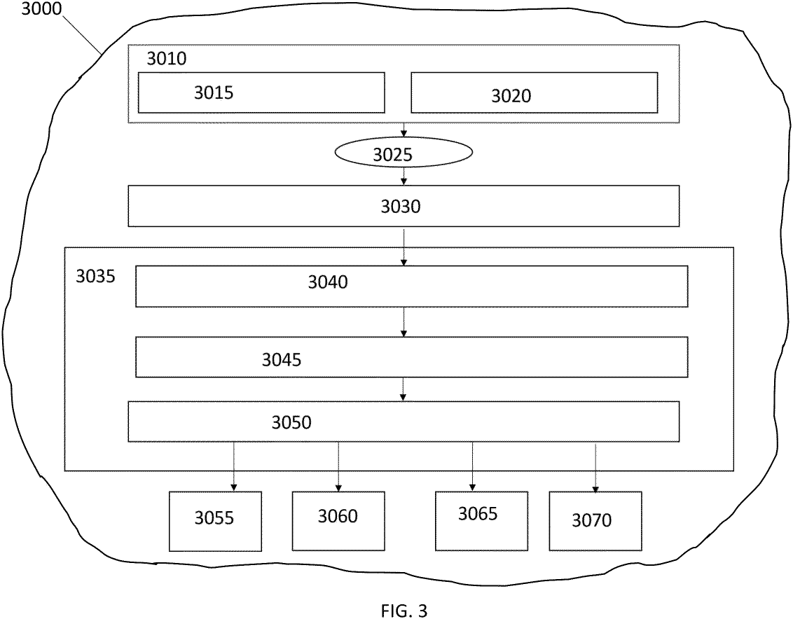

FIG. 3 shows the Web App Architecture step 3000 of the instant invention with elements of public cloud storage made up of different layers. The Web App Architecture step 3000 of FIG. 3 demonstrates a cloud-based architecture that uses Microsoft Azure Cloud. However, other embodiments may use other architectures, including non-cloud architectures, or may use cloud providers and architectures other than that provided by Microsoft Azure Cloud. The user interacts with the first layer step 3010 by way of a user device, such as a computing device with a web browser step 3015 or a mobile device such as a phone or tablet step 3020. The second layer is the load balancer step 3025, which distributes the load of inbound network traffic across the available computing resources. The third layer is the security/firewall 3030. The fourth layer, the server step 3035, has three sub-layers, namely the presentation layer step 3040, the business/application layer step 3045 and the persistent storage layer step 3050. Finally, the server step 3035 is in communication with peripherals such as data storage step 3055, third-party integration services step 3060, caching services step 3065, and other services step 3070.

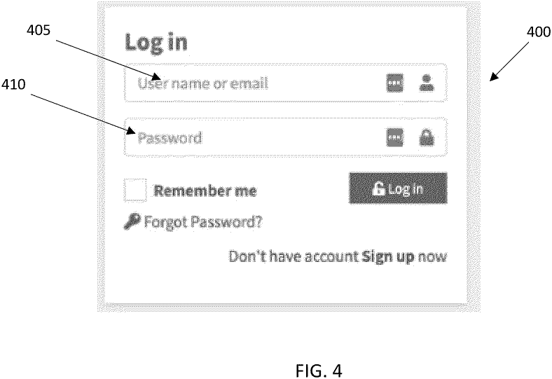

Referring to FIG. 4, is the Provider & Surgery Center Login screen 400 of the instant invention the email address 405 and password 410 is stored in in the cloud application of the instant invention

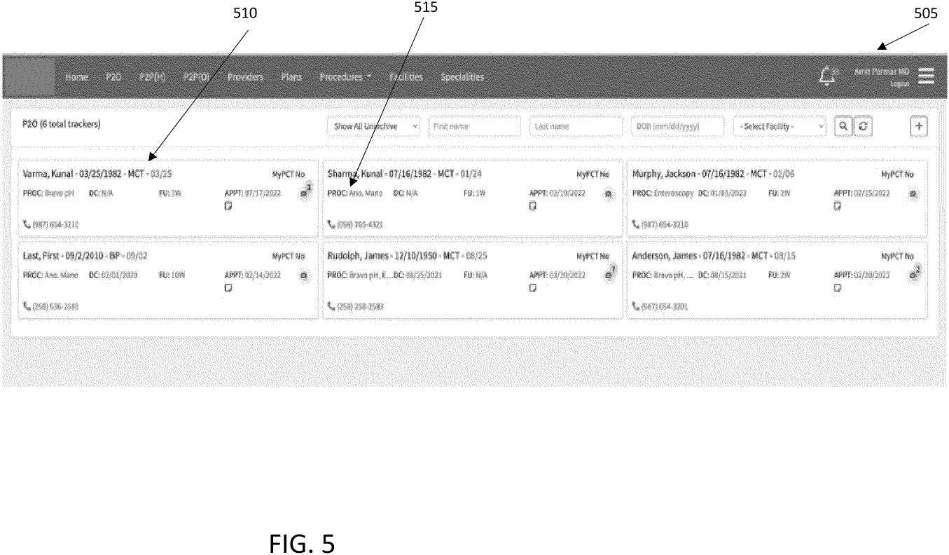

As shown in FIG. 5 is a screen view for the provider of the office tracker 505. The patient information is shown in field 510 and the procedure is shown in field 515. To access the Provider-to-Office Tracker the user selects the P2O menu item from Top Navigation bar. To create a new Provider-to-Office tracker, the user selects the plus icon on search bar.

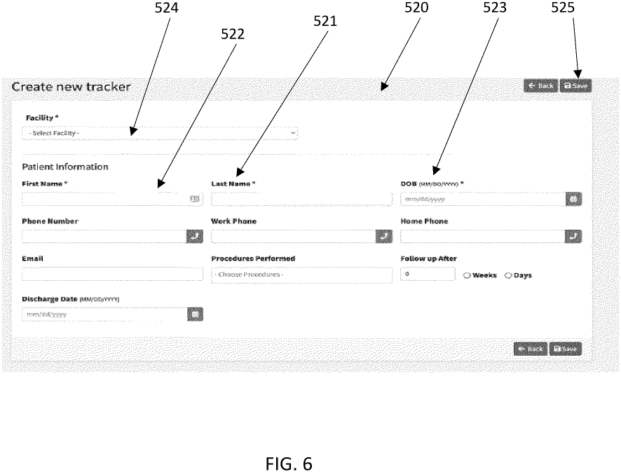

As shown in FIG. 6 provider can create a new Provider-to-Office Tracker 520. To create the new tracker the user has to input the following facility 524, first name 522, last name 521 and date of birth 523.

To save the new tracker the user selects the save button 525. To view the information the user selects the View Provider-to-Office Tracker for data and schema screen.

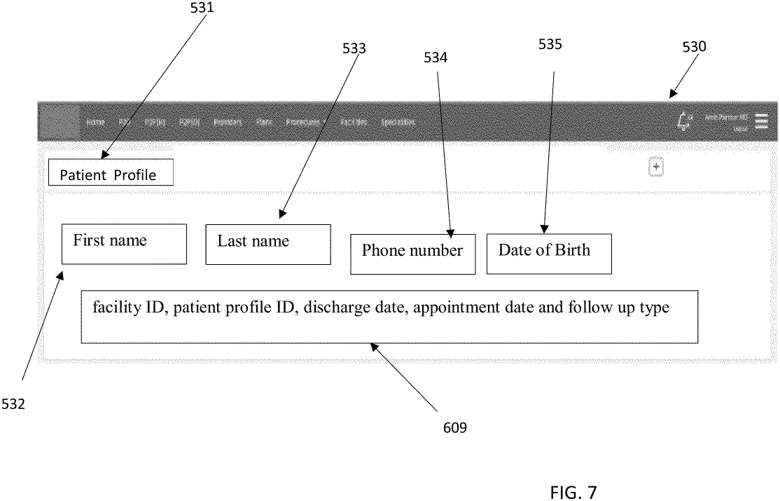

As shown in FIG. 7 the provider can View Provider-to-Office Tracker 530. The patient profile 531 contains at a minimum patients first name 532, last name 533, phone number 534 and date of birth 535.

The tracker details include at a minimum facility ID, patient profile ID, discharge date, appointment date and follow up type 609.

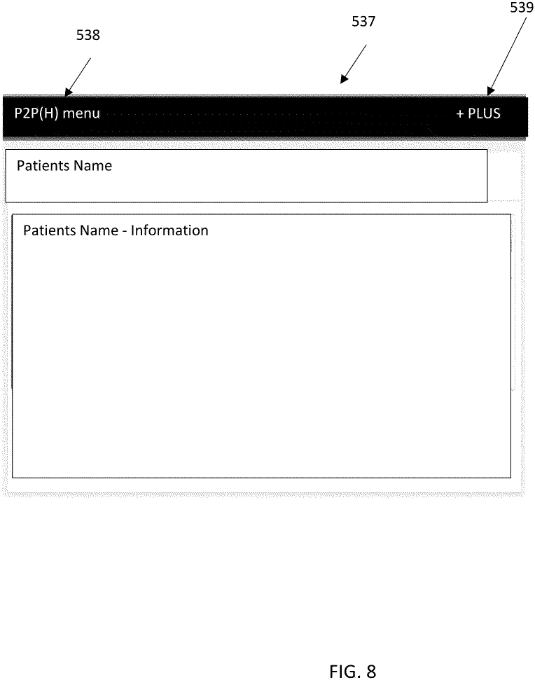

FIG. 8 shows a typical screen showing the View Provider-to-Provider - Hospital Tracker 537. To open Provider-to-Provider - Hospital Tracker the user selects the P2P(H) menu item from Top Navigation bar 538. To create new P2P tracker, select the Plus icon 539 on search bar.

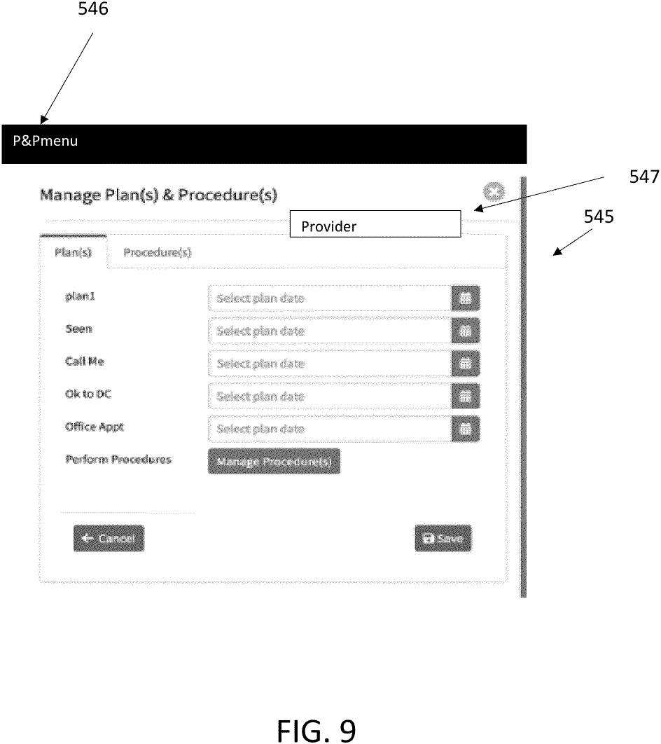

FIG. 9 shows a typical screen to showing the View Procedure(s) and Plan(s) screen 545. The user can view Plan(s) & Procedure(s) by selecting Plans & Procedure (P&P) icon 546. That will expand the List of Procedure(s) and Plan(s) and display all the applicable Procedures and Plans and the scheduled date. The tracker also contains a short name of the Provider 547 who is scheduled to perform the procedure. The instant invention uses both the P2P Office and Hospital trackers, which are similar and have the same workflow and features. The only difference is the facility.

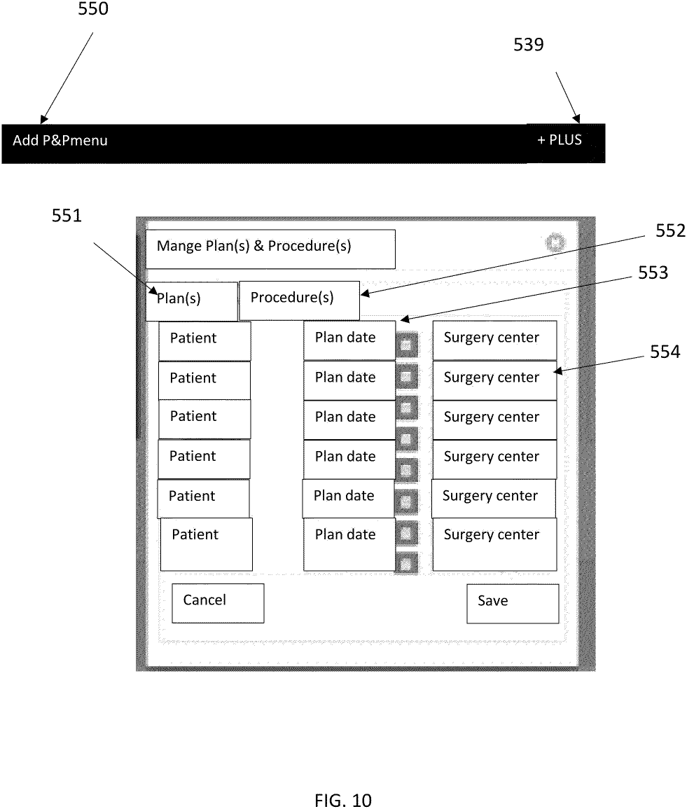

FIG. 10 shows a typical Add Procedure(s) and Plan(s) 550. The user can add plan(s) & procedure(s) by selecting the Plus Icon 539. This will open a popup window with two tabs, plan(s) 551 and procedure(s) 552. To add a plan the user enters plan date 553 for the desire plan and saves the plan. The P2P Office and Hospital tracker both have same workflow and features and the only difference is the facility.

The user adds a procedure or a plan by selecting the plus icon which opens a popup window that allows the user to input plans and procedures. An example could be that the user wants to add a procedure, the user can enter the date and then select surgery center 554 where they desire the service to be performed, and then save the transaction. The P2P Office and Hospital both have same workflow and features and the only difference is the facility information 554.

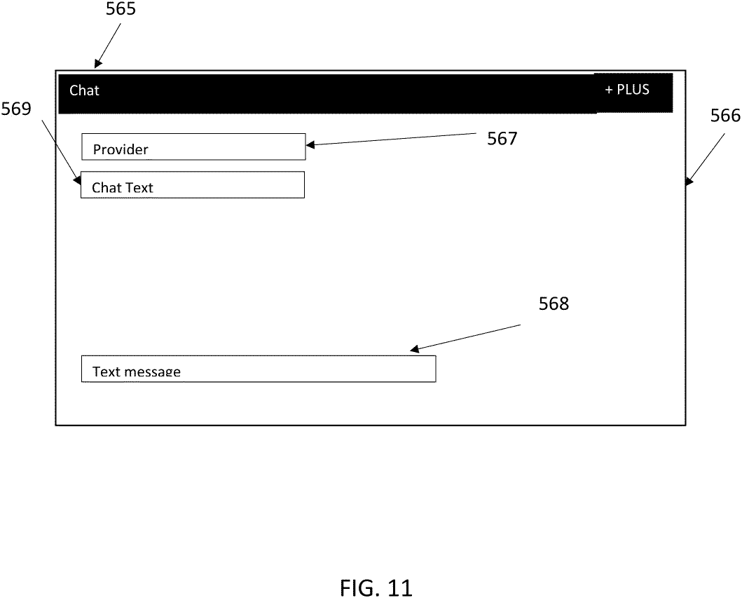

FIG. 11 shows the instant invention ability to facilitate chat/texting with a provider. To initiate a text or chat session the user selects the chat icon 565. That will open the chat window 566 and it will show the chat text with Provider details 567, Chat text 569 and Text message box 568. The chat message is encrypted as per HIPPA guideline and the system only allows sanction devices to communicate within the system. For a device to be sanctioned the device IP address must have been associated with a known authorized user such as a patient, facility or provider. The P2P Office and Hospital both have the same workflow and features, and the only difference is the facility information. The Provider Profile contains at a minimum the provider’s name and provider title which are stored in the provider profile of the instant invention. The Text Messages comprises of at least the following information: Name and sender ID, the message body and the creation time.

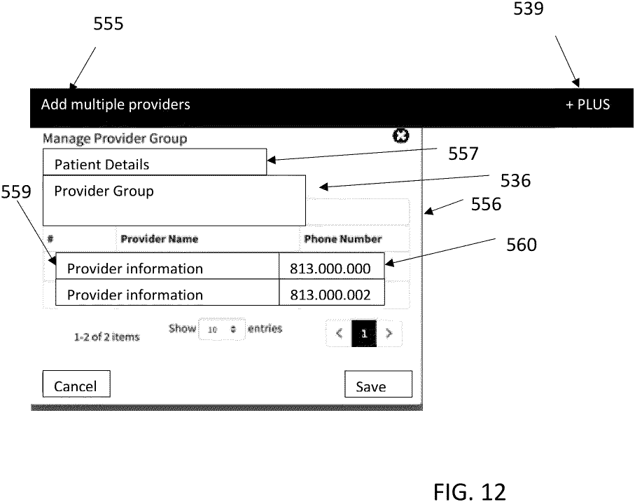

As shown in FIG. 12 the group tracker to add multiple providers is shown in screen Add multiple providers 555. To Add multiple providers the user selects the Plus Users Icon 539 which will open a Popup window 556 with Provider selection and list of added providers 559 and their phone numbers 560. The patent details 557 and provider group 536 are shown. To add a new provider the user can add the new provider by entering the information in the Provider Group Box and adding the new provider. Once a new Provider has been added to the list it cannot be removed from the list. This prevents altering the provider list. The P2P Office and Hospital both have same workflow and features; the facility is the only piece of information that differentiates the two workflows.

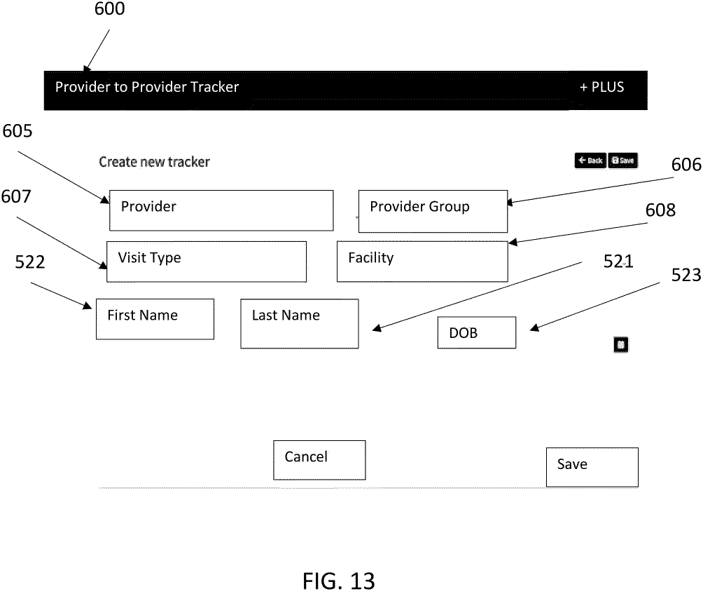

FIG. 13 shows how the instant invention creates a new provider-to-provider tracker. To create a Provider-to-Provider tracker 600, the following inputs are required.
a. Provider 605 whom you need to assign tracker.
b. Provider Group 606, if you like to add multiple providers.
c. Visit Type 607, this will differentiate Office and Hospital tracker.
d. Facility 608, it is only required when In-Patient is selected.
e. Patient First Name 522.
f. Patient Last Name 521.
g. Patient Date of Birth 523.

Patient Details are encrypted before saving to database.

The P2P Office and Hospital both have same workflow and features and are only differentiated based by facility.

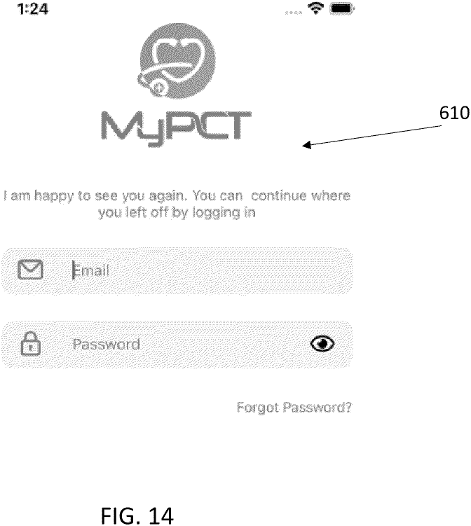

Next the instant invention provides the mobile application process. FIG. 14 provides the provider login screen 610. The process captures the email address, passwords and mobile device IP address. The IP address is verified by comparing it to the sanctioned IP address list.

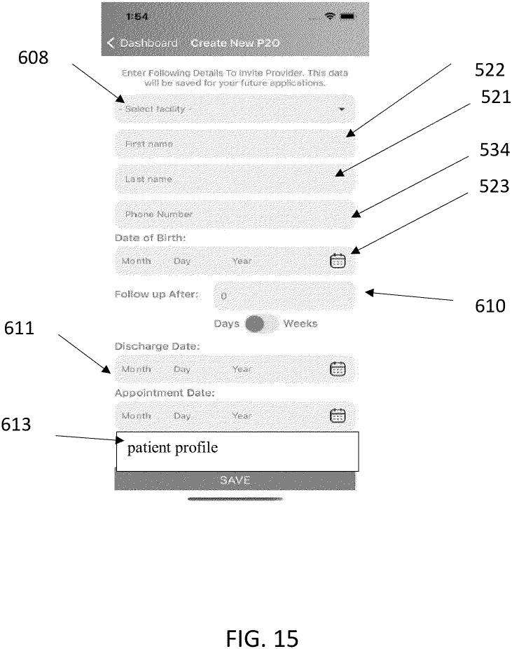

FIG. 15 shows the mobile screen for creating a Provider-to-Office Tracker. The patient profile comprises at least the following information Patient First Name 522, Patient Last Name 521, phone number 534 and Patient Date of Birth 523.

The tracker details comprise of at least the following information facility 608, patient profile ID 613, discharge date 611, appointment date 612 and follow up type 610 which is either 1 for day or 2 for week.

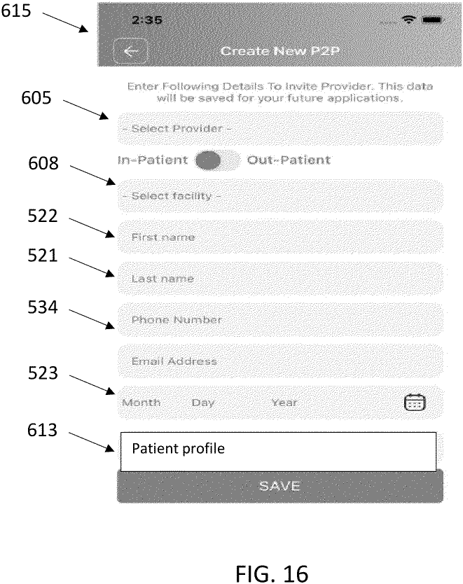

FIG. 16 shows the mobile screen for creating a Provider-to-Provider Tracker. The provider-to-provider tracker 615 has the patient profile ID 613 that is comprised of at least the following information Patient First Name 522, Patient Last Name 521, phone number 534 and Patient Date of Birth 523. The tracker details comprise of at least the following information provider 605, facility 608, patient profile ID and any other field required such as additional notes.

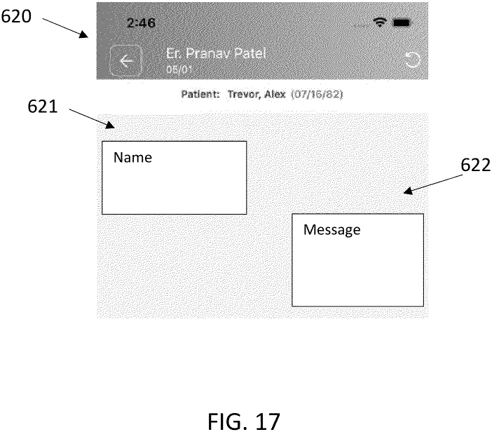

FIG. 17 shows the mobile screen for creating the Chat/Texting with Provider(s) 620. The provider profile information includes the name 621 and the text message 622 has the sender ID, message body and creation time.

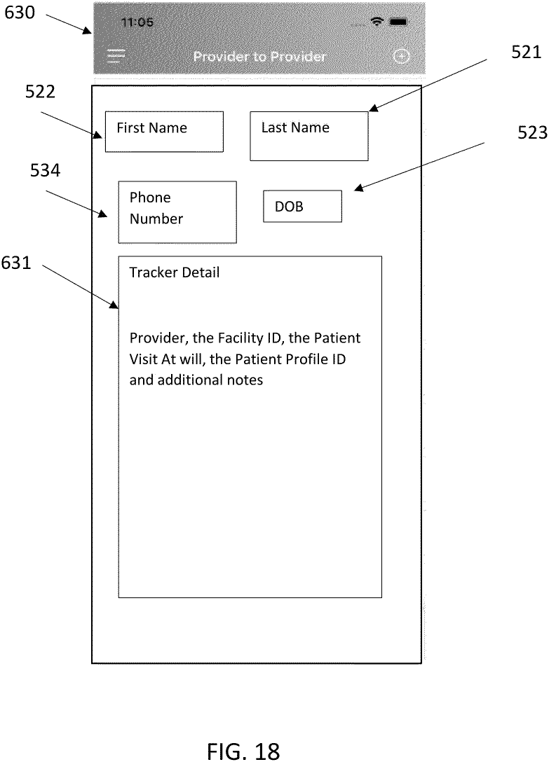

FIG. 18 shows the mobile screen for View Provider-to-Provider Information 630. The provider-to-provider tracker has the patient profile which comprises of at least the following information: Patient First Name 522, Patient Last Name 521, phone number 534 and Patient Date of Birth 523.

The tracker details 631 include at least the following information: the Provider, the Facility ID, the Patient Visit At will, the Patient Profile ID and additional notes.

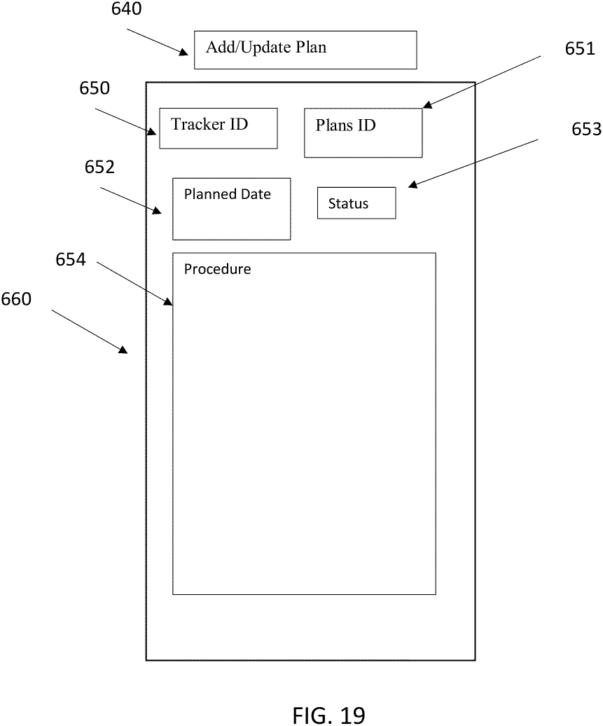

FIG. 19 shows the mobile screen to Add/Update Plan(s) 640. The information will include at least tracker ID 650, plans ID 651, planned date 652, status 653, procedure 654.

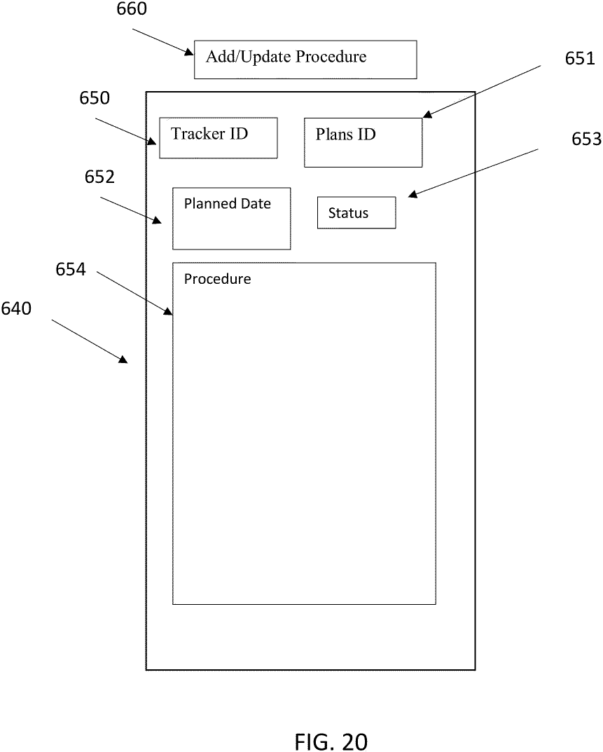

FIG. 20 shows the mobile screen to Add/Update Procedure(s) 660. The information will include at least tracker ID 650, plans ID 651, planned date 652, status 653, procedure 654.

In some embodiments, the system, method or methods described above may be executed or carried out by a computing system including a tangible computer-readable storage medium, also described herein as a storage machine, that holds machine-readable instructions executable by a logic machine such as a processor or programmable control device to provide, implement, perform, and/or enact the above described methods, processes and/or tasks. When such methods and processes are implemented, the state of the storage machine may be changed to hold different data. For example, the storage machine may include memory devices such as various hard disk drives, CD, flash drives, cloud storage, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, game controllers, video camera, camera, keyboard or gaming controller. For example, a user input may indicate a request that certain task is to be executed by the computing system, such as requesting the computing system to display any of the above described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A HIPPA compliant tracking system for a patient’s follow-up healthcare services and monitoring system comprising:
a. a provider sign up module wherein said provider sign up module collects the provider login and security information;
b. a patient sign up module wherein said patient sign up module collects the patient login and security information;
c. a patient login module wherein said provider signs in to said login module using said patient login and security information;
d. a provider login module wherein said provider signs in to said login module using said provider login and security information;
e. said HIPPA compliant tracking system collecting procedure information for at least one patient;
f. said HIPPA compliant tracking system collecting plan information for said at least one patient;
g. said HIPPA compliant tracking system displaying said procedure information for said at least one patient to at least one provider; and
h. said HIPPA compliant tracking system displaying said plan information for said at least one patient to at least one provider.

2. The HIPPA compliant tracking system of claim 1 wherein said provider sign up module stores said provider login and security information in an authorized username, password, NPI number file.

3. The HIPPA compliant tracking system of claim 2 wherein said provider login and security information comprises a username, password, an NPI number and an IP address of the computing device the provider is using.

4. The HIPPA compliant tracking system of claim 2 wherein said patient sign up module stores said patient login and security information in an authorized username, password, 10-digit alphanumeric account number file.

5. The HIPPA compliant tracking system of claim 4 wherein said provider login module compares said username, password, NPI number, to said authorized username, password, NPI number in said authorized username, password, NPI number file and said provider login module stores said IP address in a sanction IP address file if said login is a first time log in for said IP address and if said username, password, NPI number are in said authorized username, password, NPI number file.

6. The HIPPA compliant tracking system of claim 1 wherein said patient login and security information comprises of said username, password, 10-digit alphanumeric account number and an IP address of the computing device the patient is using.

7. The HIPPA compliant tracking system of claim 6 wherein said patient login and security information comprises of said username, password, 10-digit alphanumeric account number and an IP address of the computing device the provider is using.

8. The HIPPA compliant tracking system of claim 7 wherein said patient sign up module stores said patient login and security information in said authorized username, password, 10-digit alphanumeric account number file.

9. The HIPPA compliant tracking system of claim 6 wherein said patient login module compares said username, password, 10-digit alphanumeric account number to said authorized username, password, 10-digit alphanumeric account number in said authorized username, password, 10-digit alphanumeric account number file and said patient login module stores said IP address in a sanction IP address file if said login is a first time log in for said IP address and if said username, password, 10-digit alphanumeric account number are in said authorized username, password, 10-digit alphanumeric account number file.

10. The HIPPA compliant tracking system of claim 2 wherein said provider login module verifies said username, password, an NPI number and said IP address of the computing device and verifies that said IP address is in said sanctioned IP addresses and if said username, password, an NPI number is verified by the provider tracking system login verification system and said IP address is in said sanctioned IP addresses said provider tracking system login verification system allowing access to said provider tracking system.

11. The HIPPA compliant tracking system of claim 6 wherein said patient login module verifies said username, password, 10-digit alphanumeric and said IP address of the computing device and verifies that said IP address is in said sanctioned IP addresses and if said username, password, an NPI number is verified by the provider tracking system login verification system and said IP address is in said sanctioned IP addresses said provider tracking system login verification system allowing access to said provider tracking system.

12. The HIPPA compliant tracking system of claim 1 wherein said procedure information for said at least one patient comprises a medical procedure, a provider, a facility and a date of service.

13. The HIPPA compliant tracking system of claim 1 wherein said plan information for said at least one patient comprises a medical procedure, a provider, a facility and a date of service.
